(19) United States
(12) Patent Application Publication
(10) Pub. No.: US 2014/0315871 A1
(43) Pub. Date: Oct. 23, 2014

(54) TOPICAL PHARMACEUTICAL COMPOSITIONS OF ANTI-MICROBIAL AGENTS AND ANTI-INFLAMMATORY AGENTS

(71) Applicant: LUPIN LIMITED, Mumbai, Maharashtra (IN)

(72) Inventors: Raja K. Nagarajan, Pune (IN); Dhirendra Kumar, Pune (IN); Narayanan Murali, Pune (IN); Makrand Avachat, Pune (IN)

(73) Assignee: LUPIN LIMITED, Mumbai, Maharashtra (IN)

(21) Appl. No.: 14/358,956

(22) PCT Filed: Dec. 7, 2012
(86) PCT No.: PCT/IB2012/057054

§ 371 Date: May 16, 2014

(30) Foreign Application Priority Data
Dec. 9, 2011 (IN) 1534/KOL/2011

(51) Int. Cl. A61K 31/573; A61K 31/496

(52) U.S. Cl. CPC A61K 31/573; A61K 31/496
USPC 514/171

(57) ABSTRACT

The present invention provides sterile, storage-stable topical pharmaceutical compositions intended for application to the eye, ear or nose comprising Dexamethsone and Ciprofloxacin. The topical pharmaceutical compositions of invention comprises combination of ionic tonicity agent and non-ionic tonicity agent(s). The topical pharmaceutical compositions further comprises boric acid. The invention provides a process for preparing and sterilizing the compositions in order to reduce the amount of related compounds and impurities associated with the topical compositions on storage. The topical pharmaceutical compositions of invention found storage-stable, have easy resuspendability.

### FIELD OF THE INVENTION

The present invention relates to sterile, storage-stable topical pharmaceutical compositions intended for application to the eye, ear or nose comprising anti-inflammatory agent(s) and anti-microbial agent(s) and a process for preparation thereof.

### BACKGROUND OF THE INVENTION

U.S. Pat. No. 6,284,804 & U.S. Pat. No. 6,359,016 discloses topically administrable sterile ophthalmic and otic suspensions of Ciprofloxacin and Dexamethasone, marketed by Alcon under tradename CiproDex® for treatment of acute otitis media and acute otitis externa, containing ionic tonicity agent sodium chloride in an amount more than 0.3% (wt.). These suspensions are devoid of non-ionic tonicity agents like glycerine and mannitol.

U.S. Pat. No. 5,843,930 & U.S. Pat. No. 5,965,549 discloses non-ototoxic, non-irritating and non-sensitizing aqueous otic suspension for the treatment of otitis externa and otitis media, particularly otorrhea. The composition comprises Ciprofloxacin, polyvinyl alcohol, benzyl alcohol, Hydrocortisone, lecithin, polysorbate and ionic tonicity agent sodium chloride in an amount more than 0.3% (wt.) commercially marketed by Alcon under tradename CiproHC® for treatment of acute otitis externa.

U.S. Pat. No. 6,066,292 provides method for sterilizing a pharmaceutical suspension of a water-insoluble pharmaceutical like steroids including Hydrocortisone and Dexamethasone by preparation of three pre-mixes comprising i) a first sterile pre-mix of heat-sterilized aqueous solution of a viscosity enhancer (b) a second pre-mix having sterile-filtered aqueous solution of a mixture of a pharmaceutically-active compound (c) third sterile pre-mix containing heat-sterilized mixture of water, a water-insoluble pharmaceutical, and at least a partial amount of an sodium chloride to provide a sub-saturated solution, and adding under aseptic conditions an aqueous surfactant (d) combining all three pre-mixes in sterile fashion to achieve a sterile suspended pharmaceutical formulation like sterile suspension of Ciprofloxacin and Hydrocortisone.

While there are compositions available, still there remains a need to develop sterile, storage-stable topical pharmaceutical composition intended for application to the eye, ear or nose comprising anti-inflammatory agent(s) and anti-microbial agent(s) having excellent physical stability and are characterized by their easy and ready resuspendibility.

### OBJECTIVE OF THE INVENTION

The main object of the invention is to provide sterile, storage-stable topical pharmaceutical compositions intended for application to the eye, ear or nose comprising anti-inflammatory agent(s) and anti-microbial agent(s) and a process for preparation thereof.

Another object of invention is a topical pharmaceutical composition intended for application to the eye, ear or nose comprising
a. anti-inflammatory agent(s);
b. anti-microbial agent(s);
c. ionic tonicity agent(s);
d. non-ionic tonicity agent(s)
   wherein the composition has osmolality of more than 272 mOsmol/kg.

Another object of invention is a sterile topical pharmaceutical suspension composition intended for application to the eye, ear or nose comprising
a. Dexamethasone;
b. Ciprofloxacin;
c. sodium chloride;
d. glycerine;
e. boric acid
   wherein the composition optionally comprises at least one agent selected from a preservative, a pH-adjusting agent or a chelating agent.

### SUMMARY OF THE INVENTION

The present invention provides sterile, storage-stable topical pharmaceutical compositions intended for application to the eye, ear or nose comprising anti-inflammatory agent(s) and anti-microbial agent(s). The topical pharmaceutical compositions of invention comprises combination of ionic tonicity agent and non-ionic tonicity agent(s). The topical pharmaceutical compositions further comprises boric acid. The invention provides a process for preparing thereof.

### DETAILED DESCRIPTION OF THE INVENTION

The present invention relates to the sterile, storage-stable topical pharmaceutical compositions intended for application to the eye, ear or nose comprising anti-inflammatory agent(s) and anti-microbial agent(s).

The term “anti-inflammatory agent(s)” comprise but not limited to non-steroidal anti-inflammatory agents (NSAIDs) and steroids.

NSAIDs selected from Celecoxib, Rofecoxib, Meloxicam, Piroxicam, Valdecoxib, Parecoxib, Etoricoxib, Aspirin, Acetaminophen, Ibuprofen, Flurbiprofen, Ketoprofen, Naproxen, Oxaprozin, Etodolac, Bromfenac, Nepafenac, Indomethacin, Ketorolac, Lornoxicam, Nabumetone or Diclofenac or their pharmaceutically acceptable derivatives.

Steroids selected from corticosteroids comprise but not limited to Hydrocortisone, Dexamethasone or their pharmaceutically acceptable derivatives like Dexamethasone alcohol and Dexamethasone acetate.

Dexamethasone can be present in any ophthalmically or otically acceptable form having poor water solubility such that the resulting composition is a suspension composition. Suitable forms of Dexamethasone include Dexamethasone alcohol and Dexamethasone acetate. Dexamethasone alcohol is the preferred form of Dexamethasone.

The average particle size (mean volume basis) of the Dexamethasone ingredient should be less than about 25 micrometers to avoid irritation or discomfort. The average particle size is preferably less than 6 micrometers and most preferably less than 3 micrometers. Dexamethasone particles can be sized using known techniques, such as ball-milling, microfluidization, high shear homogenization, high pressure homogenization and sonication.

The term “anti-microbial agent” comprises antibiotics, fluoroquinolones selected from Ciprofloxacin, Moxifloxacin, Gatifloxacin, Norfloxacin, Perfloxacin, Amifloxacin, Pirfloxacin, Ofloxacin, Enoxacin or their pharmaceutically acceptable derivatives like polymorphs, salt, solvates, esters thereof.

Ciprofloxacin can be present in any ophthalmically or otically acceptable form such that the Ciprofloxacin is in solution in the final composition. A preferred form of Ciprofloxacin is Ciprofloxacin hydrochloride, monohydrate.

Topical pharmaceutical compositions of invention comprises but not limited to suspensions, solutions, emulsions, ointments, liniments, lotions, creams, gels, suppositories, transdermal patches, powders and osmotic pumps.

In one embodiment, a topical pharmaceutical composition intended for application to the eye, ear or nose comprising
a. anti-inflammatory agent(s);
b. anti-microbial agent(s);
c. ionic tonicity agent(s);
d. non-ionic tonicity agent(s)

Ionic tonicity agents comprises electrolytes selected from sodium chloride, potassium chloride, magnesium chloride or calcium chloride. Non-ionic tonicity agents comprises glycerine, dextrose and mannitol.

In another embodiment, a topical pharmaceutical composition intended for application to the eye, ear or nose comprises at least one tonicity agent in an amount sufficient to cause the compositions to have an osmolality of about 250-350 mOsmol/kg.

In another embodiment, a topical pharmaceutical composition intended for application to the eye, ear or nose comprises combination of one or more ionic tonicity agents in an amount sufficient to cause the compositions to have an osmolality of about 250-350 mOsmol/kg.

In another embodiment, a topical pharmaceutical composition intended for application to the eye, ear or nose comprises combination of one or more non-ionic tonicity agents in an amount sufficient to cause the compositions to have an osmolality of about 250-350 mOsmol/kg.

In another embodiment, a topical pharmaceutical composition intended for application to the eye, ear or nose comprises combination of ionic tonicity agent and non-ionic tonicity agent in an amount sufficient to cause the compositions to have an osmolality of about 250-350 mOsmol/kg.

Another object of invention is a topical pharmaceutical composition intended for application to the eye, ear or nose comprising
a. anti-inflammatory agent(s);
b. anti-microbial agent(s);
c. ionic tonicity agent(s);
d. non-ionic tonicity agent(s);
   wherein the composition has osmolality of more than about 250 mOsmol/kg, preferably more than 272 mOsmol/kg.

In another embodiment, a topical pharmaceutical composition intended for application to the eye, ear or nose comprises combination of sodium chloride and glycerine in an amount sufficient to cause the compositions to have an osmolality of about 250-350 mOsmol/kg.

In another embodiment, a topical pharmaceutical composition intended for application to the eye, ear or nose comprising
a. anti-inflammatory agent(s);
b. anti-microbial agent(s);
c. sodium chloride;
d. glycerine;
   wherein the composition has osmolality of more than about 250 mOsmol/kg, preferably more than 272 mOsmol/kg.

In another embodiment, a topical pharmaceutical composition intended for application to the eye, ear or nose comprising
a. anti-inflammatory agent(s);
b. anti-microbial agent(s);
c. ionic tonicity agent(s);
d. non-ionic tonicity agent(s)
   wherein when ionic tonicity agent is present in an amount not more than 0.3% (wt.), the composition has osmolality of more than about 250 mOsmol/kg, preferably more than 272 mOsmol/kg.

In another embodiment, a topical pharmaceutical composition intended for application to the eye, ear or nose comprising
a. anti-inflammatory agent(s);
b. anti-microbial agent(s);
c. ionic tonicity agent(s);
d. non-ionic tonicity agent(s),
   wherein when the ionic tonicity agent is sodium chloride, the composition has osmolality of more than 272 mOsmol/kg.

In another embodiment, a topical pharmaceutical composition intended for application to the eye, ear or nose comprising
a. anti-inflammatory agent(s);
b. anti-microbial agent(s);
c. ionic tonicity agent(s);
d. non-ionic tonicity agent(s),
   wherein when the ionic tonicity agent is sodium chloride, the composition has osmolality of more than 272 mOsmol/kg at a pH of 4.5±0.5; and wherein the composition optionally comprises non-ionic polymer, non-ionic surfactant, boric acid, preservative, chelating agent or buffer.

In another embodiment, a topical pharmaceutical composition intended for application to the eye, ear or nose comprising
a. Ciprofloxacin;
b. Dexamethsone;
c. ionic tonicity agent(s);
d. Glycerine,
   wherein when the ionic tonicity agent is sodium chloride, the composition has osmolality of more than 272 mOsmol/kg at a pH of 4.5±0.5; and wherein the composition optionally comprises non-ionic polymer, non-ionic surfactant, boric acid, preservative, chelating agent or buffer.

In another embodiment, a topical pharmaceutical composition intended for application to the eye, ear or nose comprising
a. anti-inflammatory agent(s);
b. anti-microbial agent(s);
c. ionic tonicity agent(s);
d. boric acid
   wherein ionic tonicity agent is present in an amount not more than 0.3% (wt.)

In another embodiment, a topical pharmaceutical composition intended for application to the eye, ear or nose comprising
a. anti-inflammatory agent(s);
b. anti-microbial agent(s);
c. ionic tonicity agent(s);
d. boric acid,
   wherein ionic tonicity agent is present in an amount not more than 0.3% (wt.) and wherein the composition optionally comprises non-ionic tonicity agent, non-ionic polymer, non-ionic surfactant, preservative, chelating agent or buffer.

In another embodiment, a topical pharmaceutical composition intended for application to the eye, ear or nose comprising
a. anti-inflammatory agent(s)
b. anti-microbial agent(s);
c. sodium chloride
d. boric acid
   wherein sodium chloride is present in an amount not more than 0.3% (wt.)

In another embodiment, a topical pharmaceutical composition intended for application to the eye, ear or nose comprising
a. anti-inflammatory agent(s);
b. anti-microbial agent(s);
c. ionic tonicity agent(s);
d. non-ionic tonicity agent(s);
e. boric acid
   wherein is ionic tonicity agent present in an amount not more than 0.3% (wt.)

Another object of invention is a topical pharmaceutical composition intended for application to the eye, ear or nose comprising
a. anti-inflammatory agent(s);
b. anti-microbial agent(s);
c. ionic tonicity agent(s);
d. non-ionic tonicity agent(s);
e. boric acid
   wherein when ionic tonicity agent is present in an amount not more than 0.3% (wt.), the composition has osmolality of more than about 250 mOsmol/kg, preferably more than 272 mOsmol/kg.

It is to be understood for the purpose of invention, the topical pharmaceutical compositions optionally comprises at least one agent selected from a preservative, a pH-adjusting agent or a chelating agent.

Preservatives comprises quaternary ammonium halide as a preservative. Suitable quaternary ammonium halides include polyquaternium-1 and benzalkonium halides. Preferred benzalkonium halides are benzalkonium chloride (“BAC”) and benzalkonium bromide. In general, the amount of the preservative ingredient will range from about 0.005-0.3% (wt.). In the preferred case where the preservative is BAC, it is preferably present at a concentration of 0.01% (wt.).

The topical pharmaceutical compositions of invention have a pH from 3-5, preferably 4.5±0.5. pH can be adjusted with NaOH/HCI. The preferred buffering system for the compositions is a combination of sodium acetate and acetic acid. The concentration of sodium acetate will generally range from 0.015-0.06% (wt.), and will preferably be about 0.03% (wt.). The concentration of acetic acid will generally range from 0.02-0.08% (wt.), and will preferably be about 0.04% (wt.).

Chelating agent comprises edetate disodium (“EDTA”); edetate trisodium; edetate tetrasodium; and diethyleneamine pentaacetate. Most preferred is EDTA. The chelating agent, if any, will typically be present in an amount from about 0.001-0.1% (wt.). In the case of EDTA, the chelating agent is preferably present at a concentration of 0.01% (wt.).

The topical pharmaceutical compositions of invention comprises one or more non-ionic polymers. These polymers include but not limited to hydroxyethyl cellulose; hydroxypropylmethyl cellulose; methyl cellulose; carboxymethyl cellulose; polyvinyl pyrrolidone and polyvinyl alcohol. The preferred non-ionic polymer is hydroxyethyl cellulose. The non-ionic polymers will be present in composition of the invention in an amount of about 0.1-0.5% (wt.). In the case of hydroxyethyl cellulose, the preferred concentration of non-ionic polymer is 0.2% (wt.).

The topical pharmaceutical compositions of invention comprises one or more non-ionic surfactant in an amount from about 0.01-0.2% (wt.). Suitable non-ionic surfactants include tyloxapol; polyoxyethylene sorbitan esters, such as polysorbate 20, polysorbate 60, and polysorbate 80; polyethoxylated castor oils, such as Cremaphor EL; polyethoxylated hydrogenated castor oils, such as HCO-40; and poloxamers. The preferred surfactant is tyloxapol.

In the case of preserved or multi-dose compositions, the topical pharmaceutical compositions of invention comprises boric acid in an amount from 0.1-1.5% (wt.).

In another embodiment, a topical suspension composition intended for application to the eye, ear or nose comprising
a. Dexamethasone;
b. Ciprofloxacin;
c. sodium chloride
d. glycerine
e. boric acid
   wherein the composition optionally comprises at least one agent selected from a preservative, a pH-adjusting agent or a chelating agent.

The Dexamethasone comprises about 0.01-0.5% (wt.) and the Ciprofloxacin comprise about 0.1-0.4% (wt.) of the composition of the invention. The preferred amounts of Dexamethasone and Ciprofloxacin in the composition of the invention are 0.1% and 0.3% (wt.), respectively.

In another embodiment, a topical suspension composition intended for application to the eye, ear or nose comprising
a) 0.1% (wt.) Dexamethasone;
b) 0.3% (wt.) Ciprofloxacin hydrochloride;
c) sodium chloride in an amount not more than 0.3% (wt.);
d) 0.6% (wt.) boric acid;
   and wherein the composition has a pH in the range of about 3 to about 5.

In another embodiment, a topical suspension composition intended for application to the eye, ear or nose comprising
a) 0.1% (wt.) Dexamethasone;
b) 0.3% (wt.) Ciprofloxacin hydrochloride;
c) sodium chloride in an amount not more than 0.3% (wt.);
d) 0.6% (wt.) boric acid;

In another embodiment, a topical suspension composition intended for application to the eye, ear or nose comprising
a) anti-inflammatory agent(s);
b) anti-microbial agent(s);
c) glycerine;
d) boric acid

Topical pharmaceutical compositions of invention can be sterilized using methods well known in the art like heat sterilization methods comprising dry heat sterilization and autoclaving (stem sterilization), gaseous sterilization methods comprising ethylene oxide sterilization, filtration sterilization methods, radiation sterilization.

In another embodiment, a method for sterilizing of a topical pharmaceutical suspension composition intended for application to the eye, ear or nose comprising
a. heat sterilizing aqueous solution of non-ionic polymer, anti-microbial agent, ionic tonicity agent, non-ionic tonicity agent to form a first sterile premix;
b. heat sterilizing mixture of non-ionic surfactant and anti-inflammatory agent to form a second sterile pre-mix;
c. combining first and second pre-mixes to form sterile topical suspension.

In another embodiment, a method for sterilizing of a topical pharmaceutical suspension composition intended for application to the eye, ear or nose comprising
a. heat sterilizing aqueous solution of hydroxyethyl cellulose, Ciprofloxacin, sodium chloride, glycerine to form a first sterile pre-mix;
b. heat sterilizing mixture of tyloxapol and Dexamethsone to form a second sterile pre-mix;
c. combining first and second pre-mixes to form sterile topical suspension.
   wherein first sterile pre-mix does not contain tyloxapol.

In another embodiment, a method for sterilizing of a topical pharmaceutical suspension composition intended for application to the eye, ear or nose comprising
a. heat sterilizing aqueous solution of non-ionic polymer, anti-microbial agent, ionic tonicity agent, non-ionic tonicity agent and non-ionic surfactant to form a first sterile pre-mix;
b. heat sterilizing anti-inflammatory agent to form a second sterile pre-mix;
c. combining first and second pre-mixes to form sterile topical suspension.

In another embodiment, a method for sterilizing of a topical pharmaceutical suspension composition intended for application to the eye, ear or nose comprising
a. heat sterilizing aqueous solution of non-ionic polymer, anti-microbial agent, ionic tonicity agent, non-ionic tonicity agent and non-ionic surfactant to form a first sterile pre-mix;
b. Ethylene oxide sterilized anti-inflammatory agent to form a second sterile pre-mix;
c. combining first and second pre-mixes to form sterile topical suspension.

In another embodiment, a method for sterilizing of a topical pharmaceutical suspension composition intended for application to the eye, ear or nose using ethylene oxide sterilization at 750 gram/m²±10% concentration of ethylene oxide having exposure time of 6.0±10% hours at minimum 50% RH±10% humidity at the temperature in the range of 40-50°±10% C.

In another embodiment, a method for sterilizing of a topical pharmaceutical suspension composition intended for application to the eye, ear or nose comprising
a. heat sterilizing aqueous solution of non-ionic polymer to form a first sterile pre-mix
b. sterile-filtering an aqueous solution of a mixture of anti-microbial compound to form a second sterile pre-mix
c. heat sterilizing anti-inflammatory agent to form third pre-mix which does not contain any electrolyte or ionic-tonicity agent like sodium chloride
d. combining all three pre-mixes under sterile condition to form a sterile topical suspension composition.

In another embodiment, a method for sterilizing a topical pharmaceutical suspension composition intended for application to the eye, ear or nose comprising
a. bulk sterilization of anti-microbial compound
b. bulk sterilization of anti-inflammatory agent

In another embodiment, a method for sterilizing a topical pharmaceutical suspension composition intended for application to the eye, ear or nose comprising
a. bulk sterilization of anti-microbial compound at 121° C. for at least 15 minutes.
b. bulk sterilization of anti-inflammatory agent at 118° C. for 30 min, at 121° C. for 20 min and at 121° C. for 15 min.

In another embodiment, a method for sterilizing a topical pharmaceutical suspension composition intended for application to the eye, ear or nose comprising
a. bulk sterilization of Ciprofloxacin at 121° C. for at least 15 minutes.
b. bulk sterilization of Dexamethsone at 118° C. for 30 min, at 121° C. for 20 min and at 121° C. for 15 min.

In another embodiment, a sterile, storage-stable topical pharmaceutical composition intended for application to the eye, ear or nose comprising
a. anti-inflammatory agent(s);
b. anti-microbial agent(s);
c. ionic tonicity agent(s);
d. non-ionic tonicity agent(s),
   wherein the composition optionally comprises at least one agent selected from a preservative, a pH-adjusting agent or a chelating agent.

In another embodiment, a topical pharmaceutical composition intended for application to the eye, ear or nose comprising
a. 0.01-0.5% (wt.) Dexamethasone;
b. 0.1-0.4% (wt.) Ciprofloxacin;
c. ionic tonicity agent(s) in an amount not more than 0.3% (wt.);
d. 0.1-1.5% (wt.) boric acid,
   wherein the composition has osmolality of about 250-350 mOsmol/kg at a pH of 4.5±0.5; and wherein the composition optionally comprises non-ionic tonicity agent, non-ionic polymer, non-ionic surfactant, preservative, chelating agent or buffer.

In another embodiment, a sterile storage-stable topical pharmaceutical suspension composition intended for application to the eye, ear or nose comprising
a. Dexamethasone;
b. Ciprofloxacin;
c. sodium chloride;
d. glycerine;
e. boric acid
   wherein the composition optionally comprises at least one agent selected from a preservative, a pH-adjusting agent or a chelating agent.

In another embodiment, a sterile topical pharmaceutical suspension composition intended for application to the eye, ear or nose comprising
a. 0.01-0.5% (wt.) Dexamethasone
b. 0.1-0.4% (wt.) Ciprofloxacin;
c. sodium chloride in an amount not more than 0.3% (wt.)
d. 0.1-5% (wt.) non-ionic tonicity agent
e. 0.1-1.5% (wt.) boric acid
f. 0.1-0.5% (wt.) non-ionic polymer
g. 0.01-0.2% (wt.) non-ionic surfactant
h. 0.005-0.3% (wt.) preservative
i. 0.001-0.1% (wt.) chelating agent
j. buffer
   wherein the composition has osmolality of about 250-350 mOsmol/kg at a pH of 4.5±0.5.

In another embodiment, a sterile topical pharmaceutical suspension composition intended for application to the eye, ear or nose comprising
a. 0.01-0.5% (wt.) Dexamethasone
b. 0.1-0.4% (wt.) Ciprofloxacin;
c. sodium chloride in an amount not more than 0.3% (wt.)
d. 0.1-5% (wt.) Glycerine
e. 0.1-1.5% (wt.) boric acid
f. 0.1-0.5% (wt.) Hydroxy Ethyl Cellulose (HEC)
g. 0.01-0.2% (wt.) Tyloxapol
h. 0.005-0.3% (wt.) Benzalkonium Chloride
i. 0.001-0.1% (wt.) Disodium Edetate
j. Sodium hydroxide/Hydrochloric Acid
   wherein the composition has osmolality of about 250-350 mOsmol/kg at a pH of 4.5±0.5.

It is to be understood for the purpose of invention that the sterile topical pharmaceutical compositions of invention can be stored in different types of containers and packaging which are well known in the art. Such types of containers and packaging keeps the sterile topical pharmaceutical compositions stable for adequate period of time.

In another embodiment, a sterile topical pharmaceutical composition of invention is “storage-stable” for period of one year under room temperature conditions and accelerated stability testing conditions like 40° C./25% RH.

In another embodiment, a topical pharmaceutical composition intended for application to the eye, ear or nose comprising
a. 0.01-0.5% (wt.) Dexamethasone;
b. 0.1-0.4% (wt.) Ciprofloxacin;
c. ionic tonicity agent(s) in an amount not more than 0.3% (wt.);
d. 0.1-1.5% (wt.) boric acid,
   wherein the composition has osmolality of about 250-350 mOsmol/kg at a pH of 4.5±0.5; and wherein the composition optionally comprises non-ionic tonicity agent, non-ionic polymer, non-ionic surfactant, preservative, chelating agent or buffer; wherein the composition comprises not more than 2.6% w/w of 20-carboxy-17-desoxy related compound when stored under conditions of 40° C./75% RH for period of 3 months.

Further sterile topical pharmaceutical suspension compositions of invention when tested for re-suspension time in “accelerated” and “real time” settling studies. The sterile topical pharmaceutical compositions of invention exhibits resuspendibility comparable to marketed composition like CiproDex®.

Topical pharmaceutical compositions of invention when subjected to preservative effectiveness test using oraganism challenge test according to the methods described in the United States Pharmacopoeia (USP) and European Pharmacopoeia (Ph. Eur.). The rate or level of anti-microbial activity determined compliance with the USP and/or Ph. Eur. preservative efficacy standards for opthalmic and/or otic preparations.

These results exhibits that the topical pharmaceutical suspension compositions of invention can be preserved such that it meets USP and/or Ph. Eur. minimum preservative efficacy requirements for opthalmic and otic compositions.

In another embodiment, invention also provides a method of treating otitis which comprises introducing an anti-bacterially effective amount of a composition as described above topically to the site of infection or inflammation. A preferred method is instilling the composition into the ear. If the ear drum is perforated, the composition can penetrate to the middle ear. Otherwise the composition can be introduced into the middle ear, for example, through a myrogotomy tube, or through the Eustachian tube by the method described in German Patent No. DE 3,617,400. To some degree, the composition can also diffuse into adjoining tissues and the middle ear when an intact ear drum is present.

In another embodiment, a sterile topical pharmaceutical composition of invention used in treating acute otitis media in patients with tympanostomy tubes due to Staphylococcus aureus, Streptococcus pneumoniae, Haemophilus influenzae, Moraxella catarrhalis, and Pseudomonas aeruginosa and acute otitis externa in patients due to Staphylococcus aureus and Pseudomonas aeruginosa.

In another embodiment, a sterile topical pharamceutical composition of invention provides a method of topically treating otitis media in patients who have open tympanic membranes. The method involves the topical application of a fixed combination of Ciprofloxacin and Dexamethasone as an aqueous suspension product. Although the dosing regimen may vary depending on the age and weight of the patient well as the severity of the infection, in most cases, the combination product would be applied twice a day. Each application would involve topically administering three or four drops into the ear canal, preferably pumping the tragus to force product through the opening in the tympanic membrane and to the site of the infection/inflammation in the middle ear.

The following examples are illustrative of the present invention, and the examples should not be considered as limiting the scope of this invention in any way, as these examples and other equivalents thereof will become apparent to those versed in the art, in the light of the present disclosure, and the accompanying claims.

### EXAMPLES

### Example 1

### Suspension Composition

| Sr. No. | Name of ingredients | % (w/v) in the composition (wt.) |
|---|---|---|
| 1 | Anti-microbial agent | 0.1-0.4 |
| 2 | Anti-inflammatory agent | 0.01-0.5 |
| 4 | Boric acid | 0.1-1.5 |
| 5 | Ionic tonicity agent | <0.3 |
| 11 | Non-ionic tonicity agent | 0.1-5 |
| 12 | Buffer | q.s to adjust pH in the rage of |
| | | 4.5 ± 0.5 |
| 14 | Purified Water | q.s. to 100 |
| | Osmolality (mOsmol/kg) | >272 |

Supension composition further comprises preservatives, chelating agents, non-ionic polymers, non-ionic surfactants.

### Example 2

### Ciprofloxacin and Dexamethasone Otic Suspension Prepared by Autoclaving

| | | % (w/v) in the |
|---|---|---|
| Sr. No. | Name of ingredients | composition (wt.) |
| 1 | Ciprofloxacin hydrochloride | 0.30 |
| | equivalent to Ciprofloxacin | |
| 2 | Dexamethasone | 0.10 |
| 3 | Benzalkonium chloride | 0.01 |
| 4 | Boric acid | 0.60 |
| 5 | Sodium chloride | 0.26 |
| 6 | Hydroxyethyl Cellulose | 0.20 |
| 7 | Tyloxapol | 0.05 |
| 8 | Acetic acid | 0.04 |
| 9 | Sodium acetate | 0.03 |
| 10 | Disodium Edetate | 0.01 |
| 11 | Glycerine | 0.75 |
| 12 | Sodium hydroxide/Hydrochloric acid | q.s to adjust pH 4.5 ± 0.5 |
| 14 | Purified Water | q.s. to 100% |
| | Osmolality (mOsmol/kg) | 297 |

### Procedure:

1. In purified water, add Hydroxyethylcellulose and dissolve with stifling.
2. In purified water, dissolve disodium edetate.
3. Dissolve Benzalkonium chloride in purified water and add to step-2.
4. Dissolve acetic acid and sodium acetate in water and add Ciprofloxacin hydrochloride and dissolve.
5. Dissolve sodium chloride in purified water and transfer to step-4.
6. Add Glycerine to step 4 with purified water.
7. Transfer step-4 to step-2.
8. Add boric acid to it and stir to dissolve.
9. Transfer step-8 to step-1 and mix the solution.
10. Adjust the pH of the solution with 0.1N Sodium hydroxide or 0.1N HC1 as required.
11. Dispense Tyloxapol in other beaker with water and dissolve in it. Disperse Dexamethasone in it.
12. Autoclave step-10 &11 at 121° C. for 15 minutes.
13. Mix step-10 solution into step-11 dispersion and makeup the volume with sterile water. Stir the suspension, then homogenize and finally stir.
14. Purge the Nitrogen in the final suspension.
15. Fill the suspension in vials under Nitrogen environment.

### Example 3

### Ciprofloxacin and Dexamethasone Otic Suspension Prepared by Autoclaving & Dry Heat Sterilization

| | | % (w/v) in the |
|---|---|---|
| Sr. No. | Name of ingredients | composition (wt.) |
| 1 | Ciprofloxacin hydrochloride | 0.30 |
| | equivalent to Ciprofloxacin | |
| 2 | Dexamethasone | 0.10 |
| 3 | Benzalkonium chloride | 0.01 |
| 4 | Boric acid | 0.60 |
| 5 | Sodium chloride | 0.26 |
| 6 | Hydroxyethyl Cellulose | 0.20 |
| 7 | Tyloxapol | 0.05 |
| 8 | Acetic acid | 0.04 |
| 9 | Sodium acetate | 0.03 |
| 10 | Disodium Edetate | 0.01 |
| 11 | Glycerine | 0.75 |
| 12 | Sodium hydroxide/Hydrochloric acid | q.s to adjust pH 4.5 ± 0.5 |
| 14 | Purified Water | q.s. to 100% |
| | Osmolality (mOsmol/kg) | 289 |

### Procedure:

1. Preparation of Hydroxyethylcellulose solution: Dissolve Hydroxyethylcellulose in purified water and stir to dissolve.
2. Preparation of Ciprofloxacin solution part:
   a. Dissolve Benzalkonium chloride, tyloxapol, sodium chloride, disodium edetate, glycerine, acetic acid, sodium acetate, boric acid and Ciprofloxacin hydrochloride in purified water stir to dissolve.
3. pH adjustment: adjust the pH if required to 4.5±0.2.
4. Volume Makeup: makeup the volume of solution with purified water.
5. Sterilization: Autoclave the step-5 solution at 121° C. for 15 minutes.
6. Nitrogen purging: Cool the step-6 solution to room temperature and purge nitrogen.
7. Dexamethasone addition: Add Dry heat sterilized Dexamethasone for CDS/920/195) with stirring to step-6 solution and continue stirring.
8. Homogenization: homogenize the step-8 suspension.
9. Mixing: finally stir.
10. Nitrogen purging: purge nitrogen in final composition.

### Example 4

### Ciprofloxacin and Dexamethasone Otic Suspension Prepared by Autoclaving & Ethylene Oxide Sterilization

Process for preparation is same as of Example 3 except ethylene oxide sterilized Dexamethasone is used in Dexamethasone addition instead of dry heat sterilized dexamethsone. Osmolality of suspension is 288 mOsmol/kg and pH is 4.5±0.5.

### Example 5

### Ciprofloxacin and Dexamethasone Otic Suspension Prepared by Autoclaving & Dry Heat Sterilization

| | | % (w/v) in the |
|---|---|---|
| Sr. No. | Name of ingredients | composition (wt.) |
| 1 | Ciprofloxacin hydrochloride | 0.30 |
| | equivalent to Ciprofloxacin | |
| 2 | Dexamethasone | 0.10 |
| 3 | Benzalkonium chloride | 0.01 |
| 5 | Sodium chloride | 0.26 |
| 6 | Hydroxyethyl Cellulose | 0.20 |
| 7 | Tyloxapol | 0.05 |
| 8 | Acetic acid | 0.04 |
| 9 | Sodium acetate | 0.03 |
| 10 | Disodium Edetate | 0.01 |
| 11 | Glycerine | 1.6 |
| 12 | Sodium hydroxide/Hydrochloric acid | q.s to adjust pH 4.5 ± 0.5 |
| 14 | Purified Water | q.s. to 100% |
| | Osmolality (mOsmol/kg) | 293 |

### Procedure:

1. Preparation of Hydroxyethylcellulose solution: Dissolve Hydroxyethylcellulose in purified water and stir to dissolve.
2. Preparation of Ciprofloxacin solution part:
   a. Dissolve Benzalkonium chloride, tyloxapol, sodium chloride, disodium edetate, glycerine, acetic acid, sodium acetate and Ciprofloxacin hydrochloride in purified water stir to dissolve.
3. pH adjustment: adjust the pH if required to 4.5±0.2.
4. Volume Makeup: makeup the volume of solution with purified water.
5. Sterilization: Autoclave the step-5 solution at 121° C. for 15 minutes.
6. Nitrogen purging: Cool the step-6 solution to room temperature and purge nitrogen.
7. Dexamethasone addition: Add Dry heat sterilized Dexamethasone for CDS/920/195) with stirring to step-6 solution and continue stirring.
8. Homogenization: homogenize the step-8 suspension.
9. Mixing: finally stir.
10. Nitrogen purging: purge nitrogen in final composition.

### Example 6

### Ciprofloxacin and Dexamethasone Otic Suspension Prepared by Autoclaving & Ethylene Oxide Sterilization

Process for preparation is same as of Example 5 except ethylene oxide sterilized Dexamethasone is used in Dexamethasone addition instead of dry heat sterilized dexamethsone. Osmolality of suspension is in the range of 250-330 mOsmol/kg and pH is 4.5±0.5.

### Resuspendibility Test—

Resuspendibility tests were performed in Centrifuge using samples of otic suspension composition of 0.3% Ciprofloxacin and 0.1% Dexamethsone of Example 2.

Accelerated settling studies were performed by subjecting otic suspension described in Example 2 in a separated glass tube for centrifugation for 30 minutes at 3100 rpm using R4C Laboratory Centrifuge (Mfg. By Remi). The resuspendibility of the settled material is tested by measuring the number of seconds of inversions required to fully re-suspend the sediment.

Real time settling studies were performed by allowing samples of Example 2 in measuring cylinder to undergo natural settling (under gravity) for seven days. The resuspendability of the settled material is tested by measuring the number of inversions required to fully re-suspend the sediment. Table 1 contains the resuspendability results of the tested sample.

TABLE 1

| | | Real-Time Settling Number | Accelarated Settling |
|---|---|---|---|
| | | of Inversions for complete | Resuspension time |
| Sr. No. | Example | resuspension | (Seconds) |
| 1 | Example 2 | 7.4 | 13.5 |

### Stability Test—

The formulation of Example 2 was found stable for period of 3 months when stored under conditions of 40° C./75% RH. The stability of Example 2 confirmed by presence of impurity 20-carboxy-17-desoxy related compound in an amount not more than 2.6% w/w.

1: A topical pharmaceutical composition intended for application to the eye, ear or nose comprising
a. anti-inflammatory agent(s);
b. anti-microbial agent(s);
c. ionic tonicity agent(s);
d. non-ionic tonicity agent(s),
wherein when the ionic tonicity agent is sodium chloride, the composition has osmolality of more than 272 mOsmol/kg at a pH of 4.5±0.5; and wherein the composition optionally comprises non-ionic polymer, non-ionic surfactant, boric acid, preservative, chelating agent or buffer.

2: The topical pharmaceutical composition according to claim 1, wherein anti-inflammatory agent(s) selected from Dexamethasone, Hydrocortisone or their pharmaceutically acceptable derivatives.

3: The topical pharmaceutical composition according to claim 1, wherein anti-microbial agent(s) selected from Ciprofloxacin, Moxifloxacin, Gatifloxacin, Norfloxacin, Perfloxacin, Amifloxacin, Pirfloxacin, Ofloxacin, Enoxacin or their pharmaceutically acceptable derivatives.

4: The topical pharmaceutical composition according to claim 1, wherein ionic tonicity agent(s) selected from sodium chloride, potassium chloride, magnesium chloride or calcium chloride.

5: The topical pharmaceutical composition according to claim 1, wherein non-ionic tonicity agent(s) selected from glycerine, dextrose or mannitol.

6: A topical pharmaceutical composition intended for application to the eye, ear or nose comprising
a. anti-inflammatory agent(s);
b. anti-microbial agent(s);
c. ionic tonicity agent(s);
d. boric acid,
wherein ionic tonicity agent is present in an amount not more than 0.3% (wt.) and wherein the composition optionally comprises non-ionic tonicity agent, non-ionic polymer, non-ionic surfactant, preservative, chelating agent or buffer.

7: A topical pharmaceutical composition intended for application to the eye, ear or nose comprising
a. 0.01-0.5% (wt.) Dexamethasone;
b. 0.1-0.4% (wt.) Ciprofloxacin;
c. ionic tonicity agent(s) in an amount not more than 0.3% (wt.);
d. 0.1-1.5% (wt.) boric acid,
wherein the composition has osmolality of about 250-350 mOsmol/kg at a pH of 4.5±0.5; and
wherein the composition optionally comprises non-ionic tonicity agent, non-ionic polymer, non-ionic surfactant, preservative, chelating agent or buffer.

8: The topical pharmaceutical composition of claim 7, wherein ionic tonicity agent(s) is sodium chloride.

9: The topical pharmaceutical composition of claim 7, is sterilized by using one or more methods selected from heat sterilization, gaseous sterilization, filtration sterilization or radiation sterilization.

10: The topical pharmaceutical composition of claim 9, comprises not more than 2.6% w/w of 20-carboxy-17-desoxy related compound when stored under conditions of 40° C./75% RH for period of 3 months.
